(19) United States
(12) Patent Application Publication
(10) Pub. No.: US 2006/0092418 A1
(43) Pub. Date: May 4, 2006

(54) Optical detection method for separating surface and deepness

(76) Inventors: Kexin Xu, Tianjin (CN); Qingjun Qiu, Tianjin (CN); Yixiong Su, Tianjin (CN)

Correspondence Address: WESTMAN CHAMPLIN & KELLY, P.A., SUITE 1400 - INTERNATIONAL CENTRE, 900 SECOND AVENUE SOUTH, MINNEAPOLIS, MN, 55402-3319, US

(21) Appl. No.: 10/528,522

(22) PCT Filed: Sep. 24, 2003
(86) PCT No.: PCT/CN03/00814

§ 371 Date: Oct. 11, 2005

(30) Foreign Application Priority Data
Sep. 29, 2002 (CN) 02129271.X

(51) Int. Cl. G01N 21/47; A61B 5/00; G01J 4/00

(52) U.S. Cl. 356/369; 356/446; 600/310

(57) ABSTRACT

This invention relates to an optical detection method for non-contact measuring an object and separating the surface and deep information of a medium in an object. A light beam that irradiates on the object from an incident unit is received by a receiving unit and detected by a detector. The separation of the surface and deep information of the medium can be achieved by a measuring system, wherein the optical probes don't contact the object. In the present invention, the incident unit and receiving unit can be configured according to polarization method, optical baffle method, space imaging method and Brewster angle method etc.

### BACKGROUND OF THE INVENTION

1. Field of the Invention

The present invention relates to an optical detection method, more particularly to an optical detection method for separating the surface and deep information of a medium in an object.

2. Description of Prior Art

Optical detection method is currently one of the most popular methods for noninvasive detection. When a light of a specific wavelength or within a specific wavelength range irradiates on a medium, due to difference in components, concentrations and particle sizes in the medium, the absorption and scattering properties of the medium will be different, and thus, the transmitted light or reflected light from the medium will possess different optical properties. Through analyzing these properties, information such as components, concentrations and particle sizes in the medium can be obtained. Such a principle enables optical detection of object components and concentrations to become more popular. Recently, noninvasive detection of human body components, particularly noninvasive detection of human blood glucose, has been attracting more and more attention. The success of noninvasive detection will help millions of patients of diabetes throughout the world release the pain and discomfort caused by invasive blood glucose detection.

Till now, among the noninvasive detection of human body components, detection method of the information in a medium includes transmission, diffuse reflection and attenuation total reflection (ATR) methods. For the transmission method, a light source and a detector are placed on the two sides of the measured position respectively, and the detector receives light transmitting through the tissue. U.S. Pat. No. 4,621,643 (New Jr., et al., 1986) is an example wherein the transmission method is applied for detecting pulse at finger tip and oxygen saturation of blood. Obviously, using transmission method, the light received by the detector represents all the information along the light propagation path. Due to the great difference between different measured individuals, even for the same individual, considerable time difference will be brought, which consequently restricts trace components inside human body from being detected by transmission method. The advantage of the diffuse reflection method is the relatively small influence of individual difference and position difference because the emitting unit and the receiving unit are placed on the same side. U.S. Pat. No. 5,028,787 (Rosenthal R. D., et al., 1991), U.S. Pat. No. 5,070,874 (Barnes R. H., et al., 1991) and Japan Patent Publication No. 8-27235 (Koashi et al., 1996) and PCT Patent WO95/06431 (Robinson M. R., 1995) etc., are good examples of the diffuse transmission method. However, the contact of the probe and measured position, contact pressure and heat conduction that causes changes to the inner structure and components distribution of the measured position, bring disturbance to the measuring results. The principle for the attenuation total reflection (ATR) is to enable multiple reactions between the sample and light so as to improve the sensitivity of output signal to the target component by using total reflection principle. In U.S. Pat. No. 4,169,676 (Kaiser N., 1979), ATR method is applied for detecting metabolites in the blood. Recently, Berman et al. (U.S. Pat. No. 6,430,424, 2002) firstly presents an invention describing a noninvasive detection method for detecting blood glucose of human body using ATR method. But for ATR method, only surface information is detected and it also requires contact detection.

In sum, non-contact measurement is the most ideal method for noninvasive detecting medium information. The most serious problem of non-contact measurement is the difficulty in separating surface and deep information of the medium. In other words, the detection of deep information needs to eliminate the disturbance of surface information. Otherwise, surface information will reach the receiving unit together with deep information, greatly influencing the accuracy of measuring result. Similarly, the detection of surface information requires the elimination of disturbance from deep information. For example, to detect the roughness of skin surface, we need to get rid of the disturbance of information of deep tissue.

### SUMMARY OF THE INVENTION

The present invention relates to a technique of optical detection method for separating surface and deep information of a medium. We present several detection methods for separating surface and deep information so as to establish a good basis for non-contact measurement.

When a light beam irradiates on a medium, e.g., the skin, from the air, the reflected light comprises of two components, as shown in FIG. 1. One of them is a direct reflected component. One study (Anderson R. R., “The optics of human skin,” J. Invest. Dermatol, 77:13-19, 1981) indicates that about 4% to 7% of the incident light reflects at the boundary because of the great difference of refractive index between the skin and air. This part of reflected light meets Fresnel law, relating to light incident angle, polarization state of the light and relative refractive index of the tissue, and the reflected light will have the same polarization state with incident light while polarized light irradiates on the medium. Furthermore, when polarized light, whose light vector is parallel to the incident plane, irradiates at Brewster angle, almost no such reflected light occurs (Liang Tingquan, Physics Optics, Mechanical Industry Publishing Office, Beijing, 1980). By analyzing the surface reflected light, we can retrieve the properties of skin surface. Another component should be backscattered light. When a light irradiates on the skin, about 93% to 96% of incident light transmits into the tissue, wherein it undergoes multiple scattering and absorption events, and part of it scatters and escapes out of the skin again and becomes part of the reflected light. The experiment shows when polarized light propagates in a turbid medium, it will finally lose its polarization property after several scattering events. And thus, when the polarized light incidents, its backscattered light has been depolarized. Because this backscattered light interacts with deep tissue, it carries plentiful information of the deep tissue. This information becomes the key part of our interest in noninvasive detection.

Based upon above principle, the present invention provides an optical detection method for separating surface and deep information of the medium, wherein the details are shown as follows.

As shown in FIG. 2, a light emitted by a light source 1 irradiates on a target sample 40 through an incident unit 2, processed by a receiving unit 3 and then detected by a detector 4.

Herein it should be emphasized that the light can irradiate on the sample 40 through a probe, and the probe doesn't directly contact the sample but non-contact. Through adjusting parameters of the incident unit and the receiving unit, separation of the surface and deep information can be achieved.

In the present invention, the incident unit and the receiving unit can be designed in different ways according to different detection methods, hereafter described respectively.

1. Polarization Method

Experiment shows that when polarized light irradiates on the skin surface, a direct reflected light from the surface keeps its polarization while the backscattered light, which penetrates into the deep tissue, undergoes multiple scattering events and escapes out of the surface, loses its polarization.

Based upon above principle, separation of the surface and deep information can be realized by applying the set-up shown in FIG. 3. In the incident unit, light is first polarized by a polarizing film 5 to transform a non-polarized light into a linearly polarized light, which is then focused on the skin surface by a focusing lens 6. In the receiving unit, the reflected light from deep tissue, together with that from the skin surface, is collected by an optical lens 7 and then is focused on a detector 9 after transmitting through a polarization analyzer 8. To receive the information of deep tissue, a polarizing film 8 is made orthogonal to the polarizing film 5, and since the backscattered light from deep tissue loses its polarization, it can reach the detector. Meanwhile, the surface reflected light keeps its polarization and can't pass through the polarizing film 8, so that the information of surface reflection is removed.

To receive surface information, the polarizing film 8 is made parallel to the polarizing film 5. Now both surface and deep information is received. As deep information is obtained under the condition of orthogonal polarization, it can be eliminated from the total reflection information received under the condition of parallel polarization, and thus, surface reflection information can be achieved.

2. Optical Baffle Method

Direct reflected light meets Fresnel law, that is, the surface reflected light of skin surface (though the surface is rough) comprises of some minor direct reflected light, and the incident point is also the reflection point. In contrast, for part of the backscattered light, there is a certain distance between the emitting point and incident point because light undergoes multiple scattering in an irregular path. And thus, the optical baffle method is used to separate the surface reflected light and backscattered light from deep tissue.

To receive information of deep tissue, influence from the surface reflected light should be removed. We use the principle shown in FIG. 4 (a). In the incident unit, an optical baffle 10 made of an opaque sheet is placed on the target position, as near as possible but non-contact. The incident and receiving light paths are positioned respectively at the two sides of the baffle, wherein the surface reflected light is at the same side with incident light so that it can be baffled by the optical baffle. In the receiving unit, reflected light from deep tissue bypasses the baffle and reflects at the receiving side, collected by a focusing lens 7 and then focused on a detector 9. And thus, light collected by the detector is all the reflected light from deep tissue so that disturbance from surface reflected light is eliminated.

To receive reflection information from the surface, the backscattered light from deep tissue should be removed. The principle is shown in FIG. 4(b). In the incident unit, an optical baffle 39 made of an opaque sheet with a very small hole in its center is placed on the target position, as near as possible but non-contact. After passing the hole, the incident light almost doesn't contain any backscattered light from deep tissue, but possesses only surface reflected light, so that disturbance from the backscattered light from deep tissue is eliminated.

3. Space Imaging Method

Space imaging method is to use geometrical optical method for separating reflected light from surface and deep tissue.

As shown in FIG. 5 (a), in the incident unit, reflection takes place at light incident point so that incident light is focused on the skin surface. In the receiving unit, according to the law of imaging, imaging point is made different from the light incident point with a distance bigger than 1 mm. An optical stop 11 is used to remove stray light. And thus, light collected by a detector 9 is all the reflected light from deep tissue and, due to imaging event, surface reflected light is unable to enter the detector, so that disturbance from the surface reflected light is eliminated. Similarly, as shown in FIG. 5 (b), when the imaging point is made as near as possible from the light incident point with a distance smaller than 1 mm, stray light being removed by the optical stop 11, light detected is almost all the surface reflected light.

4. Brewster Angle Method

According to Brewster Law, when light irradiates at Brewster angle with its polarization parallel to the incident plane, it doesn't reflect. Therefore, if the polarization state of incident light is parallel to incident plane and irradiates at Brewster angle θ_{B}, there would be no surface reflected light, so that separation of reflected light from surface and deep tissue is realized.

As shown in FIG. 6, in the incident unit, light is polarized by a polarizing film 5 so that the polarization of incident light is parallel to incident plane. After being focused by an optical lens 6, light irradiates on the skin at an incident angle approximately equal to Brewster angle of the skin surface. In the receiving unit, backscattered light is received after being focused. The imaging point of focusing light path is away from incident point as far as possible. Herein, it is particularly pointed out that Brewster angle is dependent on the wavelength of incident light: for single-wavelength measurement, Brewster angle is fixed, incident angle being set equal to Brewster angle; for multiple-wavelength measurement, Brewster angle varies with wavelength, incident angle being set as the minimum Brewster angle thereof.

### BRIEF DESCRIPTION OF THE DRAWINGS

FIG. 1 is a graph showing two components of the light reflected by the skin.

FIG. 2 is a schematic block diagram for explaining the optical detection method for separating surface and deep information of a medium.

FIG. 3 is a schematic diagram for explaining polarization method.

FIG. 4 (a) is a diagram explaining the elimination of surface reflected light using an optical baffle.

FIG. 4 (b) is a diagram explaining the elimination of reflected light from deep tissue using an optical baffle.

FIG. 5 (a) is a diagram explaining the elimination of surface reflected light using the space imaging method.

FIG. 5 (b) is a diagram explaining the elimination of reflected light from deep tissue using the space imaging method.

FIG. 6 is a schematic view of Brewster method.

FIG. 7 shows the experimental set-up of a first embodiment of the invention.

FIG. 8 is a graph for explaining energy variations of the reflected lights from the surface and the deep tissue at different incident angles.

FIG. 9 is a view of the experimental set-up for spectral measurement using the polarization method.

FIG. 10 is a graph describing spectrum of backscattered light in skin measurement using the polarization method.

FIG. 11 shows the experimental set-up for spectral measurement using the optical baffle method.

FIG. 12 shows the experimental set-up for spectral measurement using the space imaging method.

FIG. 13 is a graph describing spectrum of backscattered light in skin measurement using the space imaging method.

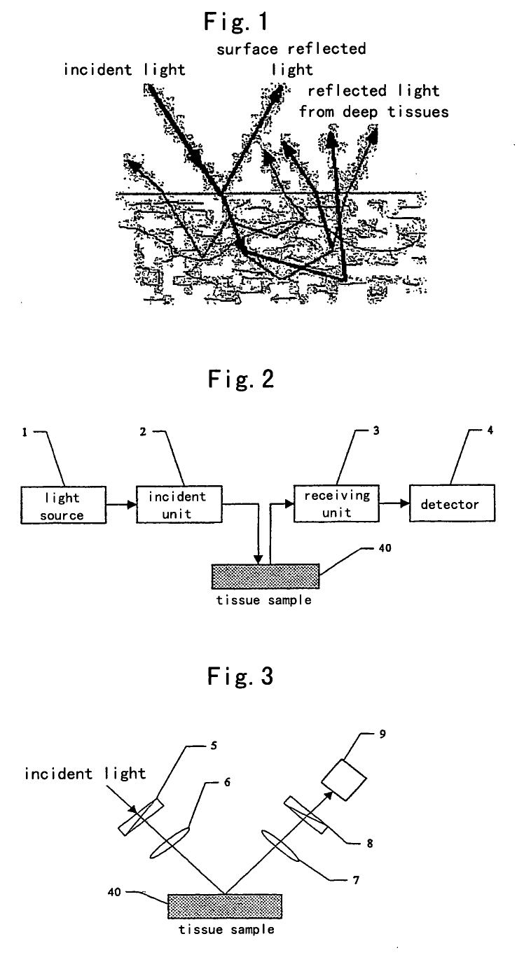

### DETAILED DESCRIPTION OF PREFERRED EMBODIMENTS

Preferred embodiments of the present invention will now be illustrated with reference to accompanying drawings and detailed embodiments.

### Embodiment 1

This experiment is designed according to above principle of the optical detection method for separating the surface and deep information of a medium. In this experiment a piece of fresh pigskin is used as the sample and the optical baffle method is applied for studying the two components, direct reflected light and backscattered light, respectively. The experimental result shows that when using linearly polarized light as the light source, direct reflected light keeps its original polarization whereas backscattered light that undergoes multiple scattering events when propagating in the tissue loses its polarization and becomes non-polarized light, and thus the principle of polarization method is proved. Furthermore, this experimental also proves the principle of optical baffle method and that of Brewster Angle method.

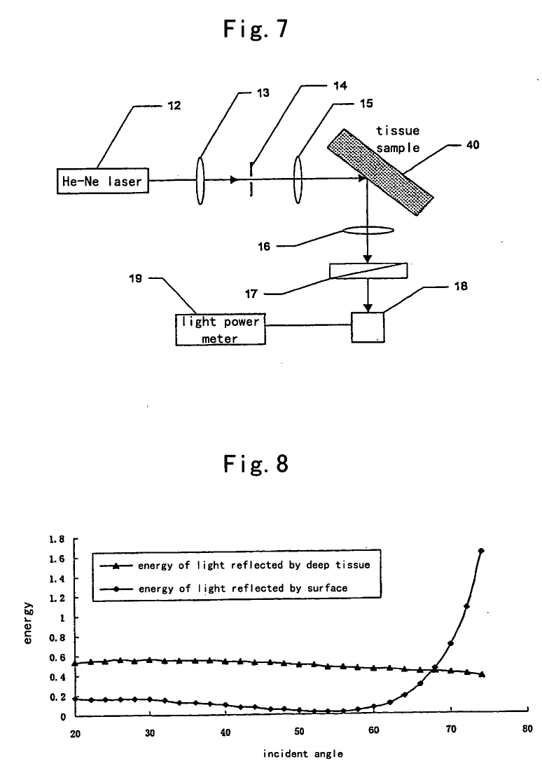

The experimental set-up is shown in the FIG. 7, a He-Ne laser 12 (type: 1101P, UNIPHASE INC.) is used as light source. This laser emits light of 632.8 nm wavelength with 4 mW output power, and its output light is a linearly polarized light whose polarization degree is 0.995. Between an optical lens 13 and an optical lens 15, an optical stop 14 is set for removing stray light caused by the laser. The light is focused on the sample after passing the optical lens 13 and 15, and thereafter the reflected light is collected by an optical lens 16, then received by an optical power meter 19 produced by NEWPORT company (type: 835), wherein the type of a probe 18 is 818 with a response frequency band ranging from 385 to 1100 nm. A polarizing film 17 is placed before the probe and is used as a polarization analyzer for detecting the polarization state of the reflected light, wherein the sample shelf can rotate round its central axis so as to adjust the incident angle of the incident light. The receiving shelf including the optical lens 16, the polarizing film 17 and the detecting probe 18 is fixed at a circular orbit with the sample shelf as its center so that the receiving angle can be adjusted conveniently.

A piece of fresh abdominal pigskin is used as the sample and is made into a sample piece sizing at 40×40 mm (area) and 10 mm (depth).

(1) Proof experiment for polarization method and optical baffle method The polarization degree is a parameter used for quantitively analyzing the polarized and non-polarized components in a light beam, which is usually defined as:

$${P}_{L} = \vert \frac{{I}_{max} - {I}_{min}}{{I}_{max} + {I}_{min}} \vert \qquad ( 1 )$$

The polarization degree P_{L} is within the range of 0-1. When P_{L} is 1, the light is a complete polarized light; when P_{L} is 0, the light is a non-polarized light; in other condition, the light becomes partly polarized light.

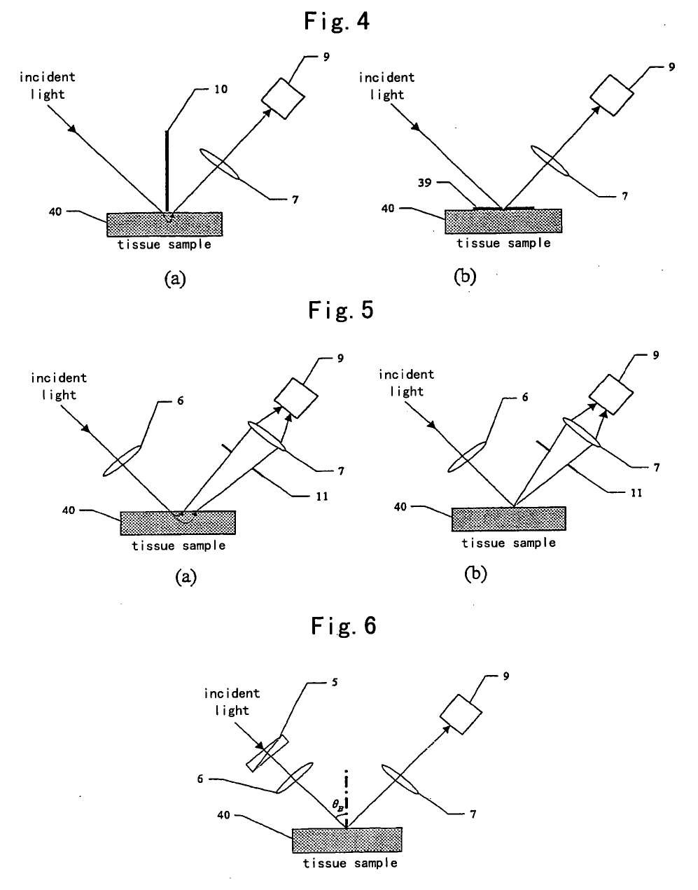

Herein the optical baffle method is used for investigating the polarization properties of surface reflected light and backscattered light from deep tissue. For study on surface reflected light, the optical baffle method is shown in FIG. 4 (b), wherein an optical baffle 39 has parameters as follows: depth, 0.2 mm; size of the central hole, 1.5 mm. When the backscattered light is under research, the optical baffle method is shown in FIG. 4 (a), wherein an optical baffle 10 being placed on the surface of a sample prevents surface reflected light from entering the detector.

Let the light irradiates at an angle of 30°. In a case of no optical baffle, the light is received at the surface reflection point, then a polarizing film 17 is rotated and Iₘₐₓ and Iₘᵢₙ are measured. Thereafter, optical baffles 39 and 10 are placed, and Iₘₐₓ and Iₘᵢₙ of the surface reflected light and backscattered light are measured, respectively. The results are shown in Table 1.

TABLE 1
Experimental results of polarization degree measurement

| | Total reflected light | Direct reflected light | Backscattered light |
|---|---|---|---|
| Iₘₐₓ | 1.66 | 1.50 | 0.045 |
| Iₘᵢₙ | 0.53 | 0.07 | 0.042 |
| P_{L} | 0.52 | 0.91 | 0.03 |

The experiment shows that there is the largest energy when the polarization state of the polarizing film 17 is parallel to that of incident light, whereas the energy becomes the smallest when the polarizing film 17 is perpendicular to that of incident light.

From the table we can see when the optical baffle 10 is placed, light received is backscattered light and its polarization degree is almost zero, and thus it can be proved that the polarized light will lose its polarization after penetrating into the tissue and undergoing multiple scattering events.

When no optical baffle is used in the experiment, light received by the optical power meter is partly polarized light with P_{L} of 0.52. Adding the optical baffle 39 prevents the backscattered light from deep tissue and results in a 75% increment of the polarization degree to 0.91. Considering the depth of the optical baffle 39 and the diameter of the central hole, influence of the backscattered light from deep tissue that completely loses its polarization state on the polarization degree can not be completely disregarded, and thus, it can be considered that the surface reflected light is linearly polarized with its polarized state parallel to that of incident light. This can just verify the feasibility of using polarization method for separating reflected light from surface and deep tissue.

Furthermore, in the optical baffle experiment for eliminating the surface reflected light, the optical baffle 10 is used in the experiment and the polarization degree of the received light is substantially zero (P_{L}=0.03), which shows the feasibility of using optical baffle method to eliminate the surface reflected light. Similarly, for eliminating the reflected light from the deep tissue, the optical baffle 39 is used, and the polarization degree of the received light is 0.91, which verifies the feasibility of using optical baffle method to eliminate the reflected light from deep tissues. Thus, this experiment verifies the feasibility of using optical baffle method for separating reflected light from surface and deep tissue.

(2) Proof Experiment for Brewster Angle Method

This experiment is mainly designed for studying the influence of Brewster angle on two reflected components from surface and deep tissue. In the experimental set-up shown in FIG. 7, the incident angle of polarized light whose polarization state is parallel to incident plane varies in the range of 20°-74°, and light is detected every 20. A polarizing film 17 is rotated and Iₘₐₓ and Iₘᵢₙ are recorded at different angles. Based upon above principle, the surface reflected light energy I_{R} can be:
I_{R}=Iₘₐₓ−Iₘᵢₙ (2)

In this proof experiment, the sample shelf and receiving shelf are rotated at the same time for adjusting the incident angle and receiving angle so that the receiving angle keeps the same with the direct reflected angle. FIG. 8 shows the experimental result. From both theoretical analysis and experimental result, it can be seen that though the skin is a complex surface, the surface reflected light meets Fresnel law. If a polarized light with its vector being parallel to incident plane irradiates on the sample surface, there also exists a Brewster angle, which is equal to about 56°, when no surface reflected light comes out. In contrast, the backscattered light from deep tissue is not affected by Brewster angle, and thus, our proof experiment verifies the feasibility of using Brewster method for separating reflected light from surface and deep tissue.

Based upon the different principles of separating surface and deep information, several experimental set-ups using non-contact method for noninvasive detection of human body components, especially for noninvasive detection of blood glucose, are established, wherein NIR spectroscopy is used, among a wavelength range of 0.8-2.5 μm, where exists absorption peak of water 6900 cm⁻¹, combination absorption spectra of glucose 4710, 4400, 4300 cm⁻¹, first order frequency multiplication absorption spectra of glucose 6200, 5920, 5775 cm⁻¹, and its second order frequency multiplication absorption spectra 960-1200 cm⁻¹.

### Embodiment 2

### Embodiment for Polarization Method

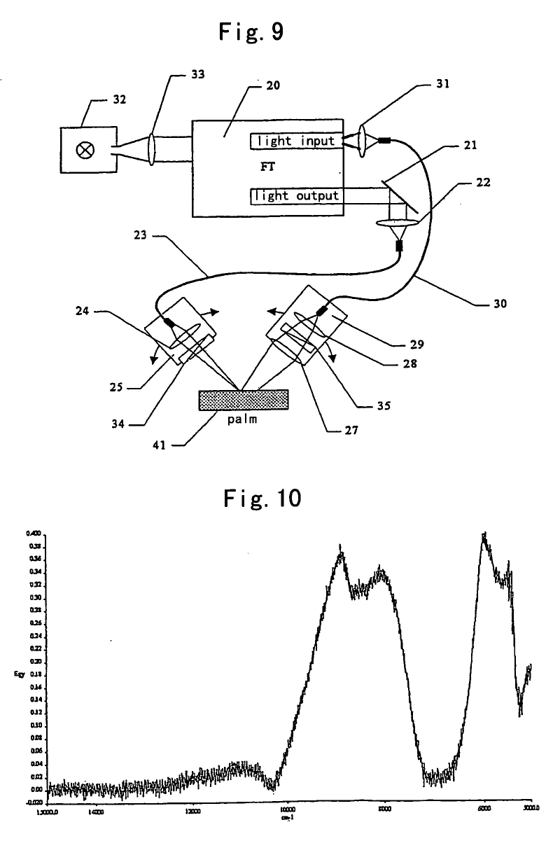

In the present embodiment, the polarization method is used for removing the surface reflected light, and non-contact spectral measurement of human body components, particularly blood glucose of human body, is achieved. The experimental set-up is shown in FIG. 9, which is carried on the palm of an object. An FT spectrometer 10 (Spectrum GX FTIR spectrometer, Perkin-Elmer Inc.) is used for spectral measurement, a 250W bromine-tungsten lamp is used as the outside light source 32, whose light is collected by an optical lens 33 to input it into the FT spectrometer. Then, it is split by the FT and passes to a reflecting mirror 21. After being coupled into an NIR light guide fiber 23 by a focusing lens 22, light is focused on the target palm when it passes an optical lens 24 and a polarizing film 34 in succession. After passing an optical lens 27, polarizing films 35 and 28, the reflected light is coupled into a light guide fiber 30, focused on the detector of FT by an optical lens 31, wherein rotation of shelf 25 and 29 is available so as to adjust the incident angle and receiving angle. The polarizing film 34 transforms incident light into linearly polarized light, its polarization state parallel to the incident plane. The polarizing film 35, whose polarization state is perpendicular to the incident plane, is used in the receiving side to remove surface reflected light.

This experimental set-up is used for spectral measurement of the palm 41, and the incident angle is 45°. Curves for describing measured spectra are shown in FIG. 10, as it is illustrated, there is almost zero energy at 6900 cm⁻¹. Because on this wavelength, water demonstrates strong absorption ability and reflected light from deep tissue has almost no energy due to water absorption, therefore, it can be explained that the light received is all backscattered light from deep tissue so that separation of reflected light from surface and deep tissue is achieved.

### Embodiment 3

### Embodiment for Optical Baffle Method

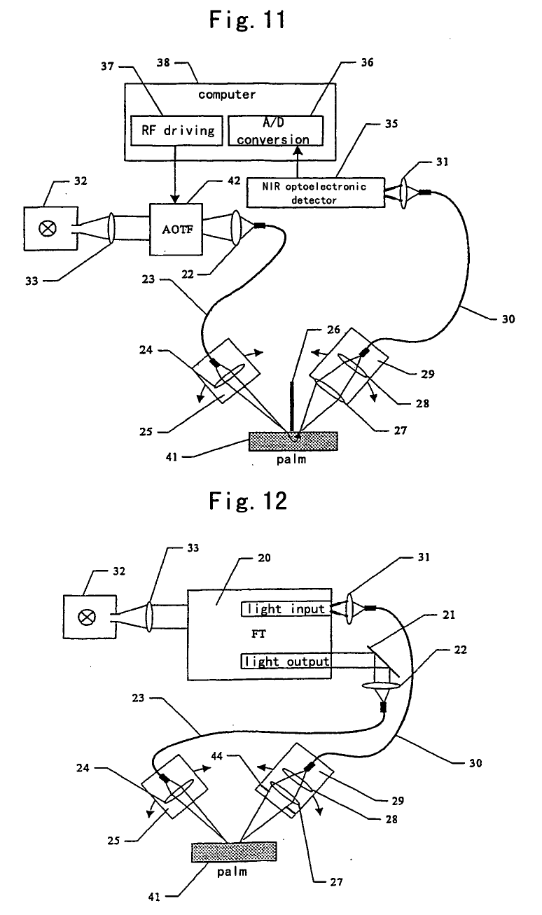

In the present embodiment, the optical baffle method is used for removing the surface reflected light, and non-contact spectral measurement of human body components, particularly blood glucose of human body, is achieved. The experimental set-up is shown in FIG. 11, where AOTF is used as the prismatic device 42, and a 250W tungsten halogen lamp is used as the outside light source 32, whose light is collected by an optical lens 33 to irradiate on the crystal of AOTF. AOTF is driven by a radio frequency driving module 37 controlled by a computer 38 for prismatic scanning of the input light. After being coupled into an NIR light guide fiber 23 by a focusing lens 22, the light is focused on the target palm 41 by an optical lens 24. An optical baffle 26 removes the surface reflected light, and after passing an optical lens 27 and a polarizing film 28, the reflected light from inner tissue is coupled into a light guide fiber 30, focused on an NIR optoelectronic detector 35 by an optical lens 31, finally collected by the computer 38 after being transformed by A/D converter. Herein the NIR optoelectronic detector could be InGaAs detector or PbS detector, and rotation of shelf 25 and 29 is available so as to adjust the incident angle and receiving angle.

Spectral measurement is performed on the same position of the palm of the same object. The measured spectrum is similar with that in FIG. 10, and therefore it can be illustrated that the light received is all the backscattered light from deep tissue so that separation of reflected light from surface and deep tissue is achieved.

### Embodiment 4

### Embodiment for Space Imaging Method

In the present embodiment, the space imaging method is used for removing surface reflected light, and non-contact spectral measurement of human body components, particularly blood glucose of human body, is achieved. The experimental set-up is shown in FIG. 12, where the FT spectrometer is also applied as a key part. Different from polarization method, no polarizing film is placed, and an optical stop 44 is used for eliminating the disturbance from stray light. For space imaging method, one requirement should be satisfied that the distance between incident point and receiving imaging point should be longer than 1 mm.

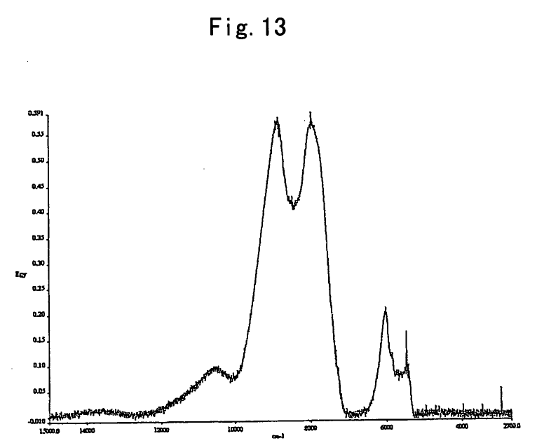

This experimental set-up is used for spectral measurement of the palm 41, and the incident angle is 45°. Curves for describing measured spectra are shown in FIG. 13, where we can see that the light received is all the backscattered light from deep tissue so that separation of reflected light from surface and deep tissue is achieved.

### Embodiment 5

### Embodiment for Brewster Angle Method

In the present embodiment, the Brewster angle method is used for removing surface reflected light, and non-contact spectral measurement of human body components, particularly blood glucose of human body, is achieved. The experimental set-up is similar with that used for polarization method, except there is no polarizing film in the receiving side. Due to the wavelength-dependence of Brewster angle, incident angle in this set-up should be adjusted a little smaller than 56° so that all wavelengths can approach Brewster angle as near as possible.

Spectral measurement is performed on the same position of the palm of the same object. The measured spectrum is similar with that in FIG. 13, and therefore it can be illustrated that a majority of light received is backscattered light from deep tissue so that separation of reflected light from surface and deep tissue is achieved.

1. An optical detection method for separating surface and deep information of a medium, wherein a light source irradiates on a measured sample through an incident unit, and the light is detected by a detector after being processed by a receiving unit, the method is characterized in that the measuring system can realize the separation of the surface and deep information; and an optical probe and the measured sample are non-contact.

2. The optical detection method for separating surface information and deep information of a medium according to claim 1, wherein a polarization method is used in said incident unit and the receiving unit; in the incident unit, a light is firstly polarized by a polarizing film to transform a non-polarized light into a linearly polarized light, which is then focused on the skin surface by a focusing lens in the receiving unit, a reflected light from deep tissue, together with that from skin surface, is collected by an optical lens and then is focused on the detector after transmitting through a polarization analyzer in order to receive the deep information of the sample, the polarizing film is made orthogonal to the polarizing film, and thus a backscattered light from a deep tissue loses its polarization so as being able to reach the detector; meanwhile, the surface reflected light keeps its polarization and can't pass through the polarizing film, so that the information of surface reflection is eliminated; in order to receive the surface information, said polarizing film is made parallel to said polarizing film, and now both said surface and deep information is received, said deep information obtained under the condition of orthogonal polarization is subtracted from the total information, and then, the surface reflection information can be achieved.

3. The optical detection method for separating surface information and deep information of a medium according to claim 1, wherein an optical baffle method is used in said incident unit and receiving unit;
in order to receive the deep information of the sample, an optical baffle is used to be perpendicularly placed over the measured sample, close to the sample as near as possible but non-contact; incident and receiving light paths are positioned respectively at the two sides of the baffle, and among the reflected light, the surface reflected light is at the same side with the incident light so that it is baffled by the optical baffle; in the receiving unit, the reflected light from deep tissue bypasses the baffle and reflects at the receiving side, collected by a focusing lens and then focused on the detector; and thus, the light collected by the detector is all the reflected light from deep tissue so that disturbance from the surface reflected light is eliminated;
in order to receive the surface reflection information, an optical baffle with a very small hole in its center is used to be perpendicularly placed over the measured sample, close the sample as near as possible but non-contact; the incident point of the incident light passes said hole, and the reflected light emitted from said hole almost doesn't contain any backscattered light from deep tissue, but possesses only the surface reflected light, so that disturbance from the deep backscattered light is eliminated.

4. The optical detection method for separating surface information and deep information of a medium according to claim 1, wherein a space imaging method is used in said incident unit and receiving unit; in the incident unit, since the reflection takes place at the light incident point, the incident light is focused on the skin surface; in the receiving unit, using a law of imaging, an imaging point is made different from the light incident point, and an optical stop is used to remove a stray light; and thus, the light collected by the detector is all the reflected light from deep tissue of the sample, and due to imaging event, the surface reflected light is unable to enter the detector, so that disturbance from the surface reflected light is eliminated; when the imaging point overlaps with the light incident point and the stray light is removed by the optical stop, the received light is almost all the light reflected by the surface of the sample.

5. The optical detection method for separating surface information and deep information of a medium according to claim 1, wherein a space imaging method is used in said incident unit and receiving unit; by using this method, a measuring device for detecting deep information of a sample can be constituted, wherein a distance between a light incident point and a receiving imaging point should be longer than lmm.

6. The optical detection method for separating surface information and deep information of a medium according to claim 1, wherein a Brewster angle method is used in said incident unit and receiving unit; in the incident unit, the light is firstly polarized by a polarizing film so that the polarization of incident light is parallel to the incident plane, after being focused by an optical lens, the light irradiates on the sample; for a single-wavelength measurement, the Brewster angle is fixed, and the incident angle is set equal to the Brewster angle; while for a multiple-wavelength measurement, the Brewster angle varies with wavelength, and the incident angle is set as the minimum Brewster angle; in the receiving unit, a backscattered light is received after being focused, and the imaging point of the focusing light path is away from the incident point as far as possible.

7. The optical detection method for separating surface information and deep information of a medium according to claim 1, wherein a measuring device for detecting sample concentration can be constituted by using any one of a polarization method, an optical baffle method, a space imaging method and a Brewster angle method; said measuring device will not be influenced by a surface reflection of the sample, and the sample is non-contact with said measuring device.

8. The optical detection method for separating surface information and deep information of a medium according to claim 1, wherein a measuring device for noninvasive detection of components of human body, especially the detection of human blood glucose concentration can be constituted by using any one of a polarization method, an optical baffle method, a space imaging method and a Brewster angle method; said measuring device will not be influenced by a surface reflection of the measured position, and the measured position is non-contact with said measuring device.

9. The optical detection method for separating surface information and deep information of a medium according to claim 1 wherein the feature comprising: an NIR spectral measuring device for detecting sample components can be constituted by using any one of a polarization method, an optical baffle method, a space imaging method and a Brewster angle method, in which a prismatic device is used for spectral measurement at any wavelength band within a range of 0.8-2.5 μm; a measuring device for detecting sample components can also be constituted, in which a laser diode emitting a single or multiple wavelength(s) is used as the light source.

10. The optical detection method for separating surface information and deep information of a medium according to claim 1, wherein said method is preferably used to construct a non-contact measuring device, and contact measurement can also be realized using said method; e.g., in an optical baffle method, the optical baffle can contact with the measured sample.

11. The optical detection method for separating surface information and deep information of a medium according to claim 1, wherein in practical application, a combination of the surface and deep information can be used; e.g., in a polarization method, when a polarization state of a polarizer is parallel to that of an analyzer, the received information contains all the deep and surface information, whereby the surface information can be obtained through calculations.
